(19) United States
(12) Patent Application Publication
(10) Pub. No.: US 2014/0289881 A1
(43) Pub. Date: Sep. 25, 2014

(54) DOUBLE-STRANDED RNA

(71) Applicant: Commonwealth Scientific and Industrial Research Organisation, Campbell (AU)

(72) Inventors: Timothy James Doran, Ocean Grove (AU); Scott Geoffrey Tyack, Grovedale (AU)

(21) Appl. No.: 14/209,007

(22) Filed: Mar. 13, 2014

Related U.S. Application Data
(60) Provisional application No. 61/783,764, filed on Mar. 14, 2013.

(51) Int. Cl. C12N 15/113; A01K 67/027

(52) U.S. Cl. CPC C12N 15/1131; A01K 67/0275; A01K 2227/30
USPC 800/19; 435/325; 435/375; 435/320.1; 514/44 A; 536/24.5; 800/13; 800/21; 800/298

(57) ABSTRACT

The present disclosure provides methods and compositions relating to a polynucleotide comprising a dsRNA region that is complementary to a particular region of the NS1 gene segment in the influenza virus genome that targets a sequence comprising two overlapping reading frames, one encoding NS1 and the second encoding the NEP polypeptide. Thus, the polynucleotide of the invention is able to target two message RNAs and is effective at inhibiting the replication of influenza virus in a cell.

### CROSS-REFERENCE TO RELATED APPLICATIONS

This application claims priority benefit of U.S. Provisional Application No. 61/783,764 filed Mar. 14, 2013, which is incorporated herein by reference in its entirety and for all purposes.

### FIELD OF THE INVENTION

The present invention relates to nucleic acid molecules comprising a double-stranded region complementary to a target gene. The invention further relates to expression vectors, cells and compositions comprising the polynucleotides, as well as methods of treating or preventing influenza in a subject by administering the polynucleotide, vector, cell or composition to the subject.

### BACKGROUND OF THE INVENTION

Three types of influenza viruses, types A, B, and C are known and they belong to a family of single-stranded negative-sense enveloped RNA viruses called Orthomyxoviridae. The viral genome is approximately 12000 to 15000 nucleotides in length and comprises eight RNA segments (seven in Type C) that encode eleven proteins.

Influenza A virus infects many animals such as humans, pigs, horses, marine mammals, and birds. Its natural reservoir is in aquatic birds, and in avian species most influenza virus infections cause mild localized infections of the respiratory and intestinal tract. However, the virus can have a highly pathogenic effect in poultry, with sudden outbreaks causing high mortality rates in affected poultry populations.

Influenza A viruses can be classified into subtypes based on allelic variations in antigenic regions of two genes that encode surface glycoproteins, namely, hemagglutinin (HA) and neuraminidase (NA) which are required for viral attachment and cellular release. Other major viral proteins include the nucleoprotein, the nucleocapsid structural protein, matrix proteins (M1 and M2), polymerases (PA, PB1 and PB2), and non-structural proteins (NS1 and NS2).

At least sixteen subtypes of HA (H1 to H16) and nine NA (N1 to N9) antigenic variants are known in influenza A virus. Avian influenza strains can also be characterized as low pathogenic and highly pathogenic strains. Low pathogenic strains typically only have two basic amino acids at positions −1 and −3 of the cleavage site of the HA precursor, while highly pathogenic strains have a multi-basic cleavage site. Subtypes H5 and H7 can cause highly pathogenic infections in poultry and certain subtypes have been shown to cross the species barrier to humans. Highly pathogenic H5 and H7 viruses can also emerge from low pathogenic precursors in domestic poultry. Symptoms of avian influenza infection range from typical influenza type symptoms (fever, cough, sore throat and muscle aches) to conjunctivitis, pneumonia, acute respiratory distress, and other life-threatening complications.

There is a need to develop ways of controlling influenza virus survival and/or replication in humans and animals such as poultry not only to improve productivity and welfare in the livestock industry, but to also reduce health risks to humans.

### SUMMARY OF THE INVENTION

The present inventors have determined that a polynucleotide comprising a dsRNA region that is complementary to a particular region of the NS1 gene segment in the influenza virus genome targets a sequence comprising two overlapping reading frames, one reading from encoding NS1 and the second encoding the NEP polypeptide. Thus, the polynucleotide of the invention is able to target two message RNAs and is particularly effective at inhibiting the replication of influenza virus in a cell.

Accordingly, the present invention provides an isolated nucleic acid molecule comprising a double-stranded region, wherein the double-stranded region comprises a sequence of nucleotides complementary to a target sequence, and wherein the target sequence is at least 90% identical to any one of SEQ ID NOs:2 to 6.

In one particular embodiment, the double-stranded region comprises a sequence of nucleotides at least 95% identical to any one of SEQ ID NOs:2 to 6.

In yet another embodiment, the double-stranded region comprises a sequence of nucleotides identical to any one of SEQ ID NOs:2 to 6.

In another embodiment, the double-stranded region comprises a sequence of nucleotides at least 95% identical to SEQ ID NO:2. In one particular embodiment, the double-stranded regions comprises a sequence of nucleotides identical to SEQ ID NO:2.

While the double-stranded region may be of any length sufficient to induce RNA interference in a cell, in one embodiment, the double-stranded region is 19 to 23 nucleotides in length. For example, the double-stranded region may be 19, 20, 21, 22 or 23 nucleotides in length.

In one embodiment, the isolated nucleic acid molecule comprises RNA or an analog thereof.

In one particular embodiment, the isolated nucleic acid molecule is an siRNA, shRNA, eshRNA, or miRNA.

In another embodiment, the isolated nucleic acid molecule further comprises one or more double-stranded regions comprising a sequence of nucleotides complementary to a target sequence in an influenza A gene. In one particular embodiment, the one or more double-stranded regions comprises a sequence of nucleotides at least 90% identical to SEQ ID NO:9 or 10.

In yet another embodiment, the isolated nucleic acid molecule reduces influenza A virus replication in an animal cell and/or reduces production of infectious influenza A virus particles in an animal cell and/or reduces the expression of an influenza A virus polypeptide in an influenza A virus infected animal cell when compared to an isogenic influenza A virus infected animal cell lacking the RNA molecule.

The present invention further provides a nucleic acid construct encoding the nucleic acid molecule of the invention.

In one embodiment, the nucleic acid construct comprises a sequence of nucleotides at least 95% identical to any one of SEQ ID NOs:7, 8 or 11.

In another embodiment, the nucleic acid construct comprises a sequence of nucleotides identical to any one of SEQ ID NOs:7, 8 or 11.

In yet another embodiment, the nucleic acid construct comprises one or more promoters. In one embodiment, the one or more promoters is an RNA polymerase III promoter. Examples of RNA polymerase III promoters include U6 and H1 promoters.

The present invention further provides a vector comprising the isolated nucleic acid molecule of the invention and/or the nucleic acid construct of the invention. Preferably, the vector is an expression vector.

The present invention further provides a cell comprising the isolated nucleic acid molecule of the invention, the nucleic acid construct of the invention, and/or the vector of the invention.

In one embodiment, the cell is an avian cell or a mammalian cell.

In another embodiment, the cell is a chicken, turkey or duck cell. In one particular embodiment the cell is a chicken primordial germ cell.

In another embodiment, the cell is a bacterial cell. For example, the cell may be a Gram negative bacterial cell, and in one particular embodiment the cell is an E. coli cell.

The present invention further provides a composition comprising the isolated nucleic acid molecule of the invention, the nucleic acid construct of the invention, the vector of the invention, and/or the cell of the invention.

In one embodiment, the composition is a pharmaceutical and/or veterinary pharmaceutical composition comprising a pharmaceutically acceptable carrier or excipient.

In another embodiment, the composition is an animal feed composition.

The present invention further provides a method of treating or preventing influenza in a subject, the method comprising administering to the subject the isolated nucleic acid molecule of the invention, the nucleic acid construct of the invention, the vector of the invention, the cell of the invention, and/or the composition of the invention.

The subject may be human. Alternatively, the subject may be a non-human animal. In one embodiment, the non-human animal is an avian. In one particular embodiment, the subject is poultry. The poultry may be, for example, a chicken, turkey or duck.

The present invention further provides a non-human transgenic organism comprising the isolated nucleic acid molecule of the invention and/or the nucleic acid construct of the invention.

In one embodiment, the non-human transgenic organism is a plant.

In another embodiment, the non-human transgenic organism is a non-human transgenic animal.

In one embodiment, the non-human transgenic animal is an avian, for example poultry. In one particular embodiment, the non-human transgenic animal is a chicken, turkey or duck.

The present invention further provides the non-human transgenic organism of the invention for use in breeding.

The present invention further provides the non-human transgenic organism of the invention for use in food production.

The present invention further provides a method of reducing the level of expression of influenza A virus NS1 and NEP genes in a cell, the method comprising introducing into the cell the isolated nucleic acid molecule of the invention, the isolated polynucleotide of the invention, the vector of the invention, the cell of the invention, and/or the composition of the invention.

In one embodiment, the level of NS1 and/or NEP mRNA in the cell is reduced in comparison to an isogenic cell that does not comprise the cell the isolated nucleic acid molecule of the invention, the isolated polynucleotide of the invention, the vector of the invention, the cell of the invention, and/or the composition of the invention.

The present invention further provides an isolated nucleic acid molecule of the invention, the isolated polynucleotide of the invention, the vector of the invention, the cell of the invention, and/or the composition of the invention for use in the treatment or prevention of influenza.

The present invention further provides use of the isolated nucleic acid molecule of the invention, the isolated polynucleotide of the invention, the vector of the invention, the cell of the invention, and/or the composition of the invention in the manufacture of a medicament for the treatment or prevention of influenza.

The present invention further provides a method of making a transgenic non-human animal, the method comprising:

(i) introducing a first nucleic acid comprising a transposon into a cell, wherein the nucleic acid encodes the isolated nucleic acid of the invention,

(ii) introducing a second nucleic acid encoding a transposase into the cell,

(ii) selecting a transgenic cell comprising the first nucleic acid in the genome of the cell,

(iii) regenerating a transgenic non-human animal from the cell, and

(iv) breeding the transgenic non-human animal.

In one embodiment, the transposon is a Tol2 transposon and the transposase is Tol2 transposase.

In another embodiment, the cell is a chicken primordial germ cell.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying Figures.

### BRIEF DESCRIPTION OF THE FIGURES

FIG. 1. HA assay to measure inhibition of virus replication. MDCK cells were transfected with shRNA plasmids and control EGFP plasmid. Cells were infected 24 hrs post transfection with H1N1 (PR8) influenza virus. Supernatant was harvested from infected cells 48 hours post infection and a HA assay performed. Results are represented as percentage inhibition compared to EGFP control (set at 100% or no inhibition).

FIG. 2. qPCR assay to measure inhibition of virus replication. MDCK cells were transfected with shRNA plasmids and control EGFP plasmid. Cells were infected 24 hrs post transfection with H1N1 (PR8) influenza virus. RNA was harvested from infected cells 48 hours post infection and a qPCR assay performed to measure viral RNA. Results were made relative to the EGFP control sample and are represented as a percentage of this control.

### KEY TO THE SEQUENCE LISTING

SEQ ID NO:1—NS1-NEP target region sequence
SEQ ID NO:2—NS1-NEP target sequence 1
SEQ ID NO:3—NS1-NEP target sequence 2
SEQ ID NO:4—NS1-NEP target sequence 3
SEQ ID NO:5—NS1-NEP target sequence 4
SEQ ID NO:6—NS1-NEP target sequence 5
SEQ ID NO:7—NS1-NEP U6 promoter construct
SEQ ID NO:8—NS1-NEP H1 promoter construct
SEQ ID NO:9—PB1-2257 siRNA target sequence
SEQ ID NO:10—PB-1498 siRNA target sequence
SEQ ID NO:11—Multi-warhead sequence
SEQ ID NO:12—Common target sequence

### DETAILED DESCRIPTION OF THE INVENTION

### General Techniques and Selected Definitions

Unless specifically defined otherwise, all technical and scientific terms used herein shall be taken to have the same meaning as commonly understood by one of ordinary skill in the art (e.g., in cell culture, molecular biology, virology, immunology, immunohistochemistry, protein chemistry, and biochemistry).

Unless otherwise indicated, the molecular biology, microbiological and cell culture techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, 3^{rd} ed., Cold Spring Harbour Laboratory Press (2001), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al., (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al., (editors) Current Protocols in Immunology, John Wiley & Sons (including all updates until present).

As used herein, the term “subject” refers to an animal, e.g., a bird or mammal. In one embodiment, the subject is a human. In other embodiments, the subject may be avian, for example poultry such as a chicken, turkey or a duck. In another embodiment, the subject is a pig.

The term “avian” as used herein refers to any species, subspecies or race of organism of the taxonomic Class Ayes, such as, but not limited to, such organisms as chicken, turkey, duck, goose, quail, pheasants, parrots, finches, hawks, crows and ratites including ostrich, emu and cassowary. The term includes the various known strains of Gallus gallus (chickens), for example, White Leghorn, Brown Leghorn, Barred-Rock, Sussex, New Hampshire, Rhode Island, Australorp, Cornish, Minorca, Amrox, Calif. Gray, Italian Partidge-coloured, as well as strains of turkeys, pheasants, quails, duck, ostriches and other poultry commonly bred in commercial quantities.

The term “poultry” includes all avians kept, harvested, or domesticated for meat or eggs, for example chicken, turkey, ostrich, game hen, squab, guinea fowl, pheasant, quail, duck, goose, and emu.

As used herein the terms “treating”, “treat” or “treatment” include administering a therapeutically effective amount of a nucleic acid construct, vector, cell and/or nucleic acid molecule of the invention sufficient to reduce or eliminate at least one symptom of an influenza A virus infection, especially avian influenza virus, infection.

The term “preventing” refers to protecting a subject that is exposed to influenza A virus from developing at least one symptom of an influenza A virus infection, or reducing the severity of a symptom of infection in a subject exposed to influenza A virus.

The influenza virus may be an influenza A virus. The influenza A virus may be selected from influenza A viruses isolated from avian and mammalian organisms. In particular, the influenza A virus may be selected from H1N1, H1N2, H1N3, H1N4, H1N5, H1N6, H1N7, H1N9, H2N₁, H2N₂, H2N₃, H2N₄, H2N₅, H2N₇, H2N₈, H2N₉, H3N1, H3N2, H3N3, H3N4, H3N5, H3N6, H3N8, H4N1, H4N2, H4N3, H4N4, H4N5, H4N6, H4N8, H4N9, H5N1, H5N2, H5N3, H5N6, H5N7, H5N8, H5N9, H6N1, H6N2, H6N3, H6N4, H6N5, H6N6, H6N7, H6N8, H6N9, H7N1, H7N2, H7N3, H7N4, H7N5, H7N7, H7N8, H7N9, H9N1, H9N2, H9N3, H9N5, H9N6, H9N7, H9N8, H10N1, H10N3, H10N4, H10N6, H10N7, H10N8, H10N9, H11N2, H11N3, H11N6, H11N9, H12N1, H12N4, H12N5, H12N9, H13N2, H13N6, H13N8, H13N9, H14N5, H15N2, H15N8, H15N9 and H16N3. In one embodiment, the influenza A virus is selected from H1N1, H3N2, H7N7, and/or H5N1.

“Virus replication” as used herein refers to the amplification of the viral genome in a host cell.

By “reduces the expression of” or “reducing the expression of” a polypeptide, polynucleotide or gene is meant that the translation of a polypeptide sequence and/or transcription of a polynucleotide sequence in a host cell is down-regulated or inhibited. The degree of down-regulation or inhibition will vary with the nature and quantity of the nucleic acid construct or nucleic acid molecule provided to the host cell, the identity, nature, and level of nucleic acid molecule(s) of the invention expressed from the construct, the time after administration, etc., but will be evident e.g., as a detectable decrease in target gene/sequence protein expression and/or related target or cellular function, or e.g., decrease in level of viral replication, etc.; desirably a degree of inhibition greater than 10%, 33%, 50%, 75%, 90%, 95% or 99% as compared to a cell not treated according to the present invention will be achieved.

As used herein, the term “transposon” refers to a genetic element that can move (transpose) from one position to another within the genome of an organism by processes which do not require extensive DNA sequence homology between the transposon and the site of insertion nor the recombination enzymes need for classical homologous crossing over.

As used herein, the term “introducing” as it relates to a nucleic acid construct or nucleic acid molecule is to be taken in the broadest possible sense and include any method resulting in the nucleic acid construct or nucleic acid molecule being present in a cell or organism. For example, the nucleic acid construct or nucleic acid molecule may be delivered to a cell as naked DNA via any suitable transfection or transformation technique such as, for example, electroporation. Alternatively, the nucleic acid construct or nucleic acid molecule may be inserted into the genome and/or be expressed by a transgene in a cell.

As used herein, “isogenic” refers to organisms or cells that are characterised by essentially identical genomic DNA, for example the genomic DNA is at least about 92%, preferably at least about 98%, and most preferably at least about 99%, identical to the genomic DNA of an isogenic organism or cell.

### Nucleic Acids and RNA Interference

The terms “RNA interference”, “RNAi” or “gene silencing” are well known in the art and refer generally to a process in which a double-stranded RNA molecule reduces the expression of a target nucleic acid sequence with which the double-stranded RNA molecule shares substantial or total sequence identity. It has been shown that RNA interference can be achieved using non-RNA double stranded molecules (see, for example, US 20070004667).

The present invention includes nucleic acid molecules comprising and/or encoding double-stranded regions for RNA interference. The nucleic acid molecules are typically RNA but may comprise chemically-modified nucleotides and non-nucleotides.

The double-stranded regions should be at least 19 contiguous nucleotides, for example about 19 to 23 nucleotides, or may be longer, for example 30 or 50 nucleotides, or 100 nucleotides or more. The full-length sequence corresponding to the entire gene transcript may be used.

The degree of identity of a double-stranded region of a nucleic acid molecule to the targeted transcript should be at least 90% and more preferably 95-100%. The nucleic acid molecule may of course comprise unrelated sequences which may function to stabilize the molecule.

The nucleic acid molecules of the present invention may be siRNA, shRNA, miRNA, short interfering nucleic acid (siNA), short interfering modified oligonucleotide, chemically-modified siRNA, post-transcriptional gene silencing RNA (ptgsRNA), and others.

The term “short interfering RNA” or “siRNA” as used herein refers to a nucleic acid molecule which comprises ribonucleotides capable of inhibiting or down regulating gene expression, for example by mediating RNAi in a sequence-specific manner, wherein the double stranded portion is less than 50 nucleotides in length, preferably about 19 to about 23 nucleotides in length. For example the siRNA can be a nucleic acid molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siRNA can be assembled from two separate oligonucleotides, where one strand is the sense strand and the other is the antisense strand, wherein the antisense and sense strands are self-complementary.

By “shRNA” or “short-hairpin RNA” is meant an RNA molecule where less than about 50 nucleotides, preferably about 19 to about 23 nucleotides, is base paired with a complementary sequence located on the same RNA molecule, and where said sequence and complementary sequence are separated by an unpaired region of at least about 4 to about 15 nucleotides which forms a single-stranded loop above the stem structure created by the two regions of base complementarity. An example of a sequence of a single-stranded loop includes: 5′ UUCAAGAGA 3′. In one embodiment, the nucleic acid molecule is an extended shRNA (“eshRNA”) that can be processed by the RNAi machinery into multiple siRNA duplexes (Liu et al., (2007)). An eshRNA construct typically comprises a single promoter, two or three sequences encoding siRNA sequences targeting a gene of interest and a loop sequence.

Included shRNAs are dual or bi-finger and multi-finger hairpin dsRNAs, in which the RNA molecule comprises two or more of such stem-loop structures separated by single-stranded spacer regions.

MicroRNAs (miRNAs) are small single-stranded non-coding RNAs that play critical roles in the regulation of biological processes. MicroRNAs are initially transcribed as a long, single-stranded miRNA precursor known as a primary-miRNA (pri-miRNA), which may contain one or several miRNAs. These pri-miRNAs typically contain regions of localized stem-loop hairpin structures that contain the mature miRNA sequences. Pri-miRNAs are processed into 70-100 nucleotide pre-miRNAs in the nucleus by the double-stranded RNA-specific nuclease Drosha. These 70-100 nucleotide pre-miRNAs are transported to the cytoplasm, where they are processed by the enzyme Dicer into single-stranded mature miRNAs of about 19-25 nucleotides. As known in the art, naturally-occurring or synthetic miRNAs may be modified to comprise a sequence of nucleotides complementary to one or more target gene sequences of interest.

Once designed, the nucleic acid molecules comprising a double-stranded region can be generated by any method known in the art, for example, by in vitro transcription, recombinantly, or by synthetic means.

Modifications or analogs of nucleotides can be introduced to improve the properties of the nucleic acid molecules of the invention. Improved properties include increased nuclease resistance and/or increased ability to permeate cell membranes. Accordingly, the terms “nucleic acid molecule” and “double-stranded RNA molecule” includes synthetically modified bases such as, but not limited to, inosine, xanthine, hypoxanthine, 2-aminoadenine, 6-methyl-, 2-propyl- and other alkyl-adenines, 5-halo uracil, 5-halo cytosine, 6-aza cytosine and 6-aza thymine, pseudo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

As known in the art, RNAi molecules such as siRNA and shRNA may contain a mismatch, referred to as a “bulge”, in the antisense strand in order to reduce or eliminate off-target silencing (Dua et al., 2011). For example, a nucleic acid molecule of the invention comprising a double-stranded region of 19 nucleotides in length may comprise a single non-matching nucleotide near or at the 5′ end of the molecule. Thus, despite the antisense (guide) strand of the double-stranded region comprising 20 nucleotides and the sense strand comprising 19 nucleotides, such nucleic acid molecules of the invention are still be considered to comprise a double-stranded region of 19 nucleotides. Similarly, a limited number of mismatches may be introduced into the sense (passenger) strand without limiting the effectiveness of the RNAi molecule.

### Nucleic Acid Molecules

By “isolated nucleic acid molecule” we mean a nucleic acid molecule which has generally been separated from the nucleotide sequences with which it is associated or linked in its native state (if it exists in nature at all). Preferably, the isolated nucleic acid molecule is at least 60% free, more preferably at least 75% free, and more preferably at least 90% free from other components with which it is naturally associated. Furthermore, the term “nucleic acid molecule” is used interchangeably herein with the term “polynucleotide”.

The terms “nucleic acid molecule” or “polynucleotide” refer to an oligonucleotide, polynucleotide or any fragment thereof. It may be DNA or RNA of genomic or synthetic origin, and combined with carbohydrate, lipids, protein, or other materials to perform a particular activity defined herein.

The % identity of a nucleic acid molecule is determined by GAP (Needleman and Wunsch, 1970) analysis (GCG program) with a gap creation penalty=5, and a gap extension penalty=0.3. The query sequence is at least 19 nucleotides in length, and the GAP analysis aligns the two sequences over a region of at least 19 nucleotides. Alternatively, the query sequence is at least 150 nucleotides in length, and the GAP analysis aligns the two sequences over a region of at least 150 nucleotides. Alternatively, the query sequence is at least 300 nucleotides in length and the GAP analysis aligns the two sequences over a region of at least 300 nucleotides. Preferably, the two sequences are aligned over their entire length.

With regard to the defined nucleic acid molecules, it will be appreciated that % identity figures higher than those provided above will encompass preferred embodiments. Thus, where applicable, in light of the minimum % identity figures, it is preferred that the nucleic acid molecule comprises a nucleotide sequence which is at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

A nucleic acid molecule of the present invention may selectively hybridise to a polynucleotide that encodes an influenza A virus polypeptide under stringent conditions. As used herein, under stringent conditions are those that (1) employ low ionic strength and high temperature for washing, for example, 0.015 M NaCl/0.0015 M sodium citrate/0.1% NaDodSO4 at 50° C.; (2) employ during hybridisation a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS and 10% dextran sulfate at 42° C. in 0.2×SSC and 0.1% SDS.

Usually, monomers of a nucleic acid are linked by phosphodiester bonds or analogs thereof to form oligonucleotides ranging in size from a relatively short monomeric units, e.g., 19-25, to several hundreds of monomeric units. Analogs of phosphodiester linkages include: phosphorothioate, phosphorodithioate, phosphoroselenoate, phosphorodiselenoate, phosphoroanilothioate, phosphoranilidate, phosphoramidate.

### Nucleic Acid Constructs

As used herein, “nucleic acid construct” refers to any nucleic acid molecule that encodes a double-stranded RNA molecule as defined herein and includes the nucleic acid molecule in a vector, the nucleic acid molecule when present in a cell as an extrachromosomal nucleic acid molecule, and a nucleic acid molecule that is integrated into the genome. Typically, the nucleic acid construct will be double stranded DNA or double-stranded RNA, or a combination thereof. Furthermore, the nucleic acid construct will typically comprise a suitable promoter operably linked to the open reading frame encoding the double-stranded RNA. The nucleic acid construct may comprise a first open reading frame encoding a first single strand of the double-stranded RNA molecule, with the complementary (second) strand being encoded by a second open reading frame by a different, or preferably the same, nucleic acid construct. The nucleic acid construct may be a linear fragment or a circular molecule and it may or may not be capable of replication. The skilled person will understand that the nucleic acid construct of the invention may be included within a suitable vector. Transfection or transformation of the nucleic acid construct into a recipient cell allows the cell to express an RNA molecule encoded by the nucleic acid construct.

The nucleic acid construct of the invention may express multiple copies of the same, and/or one or more (e.g. 1, 2, 3, 4, 5, or more) including multiple different, RNA molecules comprising a double-stranded region, for example a short hairpin RNA. RNA molecules considered to be the “same” as each other are those that comprise only the same double-stranded sequence, and RNA molecules considered to be “different” from each other will comprise different double-stranded sequences, regardless of whether the sequences to be targeted by each different double-stranded sequence are within the same, or a different gene, or sequences of two different genes.

The nucleic acid construct also may contain additional genetic elements. The types of elements that may be included in the construct are not limited in any way and may be chosen by one with skill in the art. In some embodiments, the nucleic acid construct is inserted into a host cell as a transgene. In such instances it may be desirable to include “stuffer” fragments in the construct which are designed to protect the sequences encoding the RNA molecule from the transgene insertion process and to reduce the risk of external transcription read through. Stuffer fragments may also be included in the construct to increase the distance between, e.g., a promoter and a coding sequence and/or terminator component. The stuffer fragment can be any length from 5-5000 or more nucleotides. There can be one or more stuffer fragments between promoters. In the case of multiple stuffer fragments, they can be the same or different lengths. The stuffer DNA fragments are preferably different sequences. Preferably, the stuffer sequences comprise a sequence identical to that found within a cell, or progeny thereof, in which they have been inserted. In a further embodiment, the nucleic acid construct comprises stuffer regions flanking the open reading frame(s) encoding the double stranded RNA(s).

Alternatively, the nucleic acid construct may include a transposable element, for example a transposon characterized by terminal inverted repeat sequences flanking the open reading frames encoding the double stranded RNA(s). Examples of suitable transposons include Tol2, mini-Tol, Sleeping Beauty, Mariner and Galluhop.

Other examples of an additional genetic element which may be included in the nucleic acid construct include a reporter gene, such as one or more genes for a fluorescent marker protein such as GFP or RFP; an easily assayed enzyme such as beta-galactosidase, luciferase, beta-glucuronidase, chloramphenical acetyl transferase or secreted embryonic alkaline phosphatase; or proteins for which immunoassays are readily available such as hormones or cytokines. Other genetic elements that may find use in embodiments of the present invention include those coding for proteins which confer a selective growth advantage on cells such as adenosine deaminase, aminoglycodic phosphotransferase, dihydrofolate reductase, hygromycin-B-phosphotransferase, or drug resistance.

Where the nucleic acid construct is to be transfected into an animal, it is desirable that the promoter and any additional genetic elements consist of nucleotide sequences that naturally occur in the animal's genome. It is further desirable that the sequences encoding RNA molecules consist of influenza A virus sequences.

### Vectors and Host Cells

In some instances it may be desirable to insert the nucleic acid construct and/or nucleic acid molecule of the invention into a vector. The vector may be, e.g., a plasmid, virus or artificial chromosome derived from, for example, a bacteriophage, adenovirus, adeno-associated virus, retrovirus, poxvirus or herpesvirus. Such vectors include chromosomal, episomal and virus-derived vectors, e.g., vectors derived from bacterial plasmids, bacteriophages, yeast episomes, yeast chromosomal elements, and viruses, vectors derived from combinations thereof, such as those derived from plasmid and bacteriophage genetic elements, cosmids and phagemids. Thus, one exemplary vector is a double-stranded DNA phage vector. Another exemplary vector is a double-stranded DNA viral vector.

The vector into which the nucleic acid construct is inserted may also include a transposable element, for example a transposon characterized by terminal inverted repeat sequences flanking the open reading frames encoding the double stranded RNA(s). Examples of suitable transposons include Tol2, Mini-Tol2, Sleeping Beauty, Mariner and Galluhop. Reference to a Tol2 tansposon herein includes a transposon derived from Tol2 such as Mini-Tol2.

The present invention also provides a host cell into which the nucleic acid construct, nucleic acid molecule and/or the vector of the present invention has been introduced. The host cell of this invention can be used as, for example, a production system for producing or expressing the dsRNA molecule. For in vitro production, eukaryotic cells or prokaryotic cells can be used.

Useful eukaryotic host cells may be animal, plant, or fungal cells. As animal cells, mammalian cells such as CHO, COS, 3T3, DF1, CEF, MDCK myeloma, baby hamster kidney (BHK), HeLa, or Vero cells, amphibian cells such as Xenopus oocytes, or insect cells such as Sf9, Sf21, or Tn5 cells can be used. CHO cells lacking DHFR gene (dhfr-CHO) or CHO K−1 may also be used. The vector can be introduced into the host cell by, for example, the calcium phosphate method, the DEAE-dextran method, cationic liposome DOTAP (Boehringer Mannheim) method, electroporation, lipofection, etc.

Useful prokaryotic cells include bacterial cells, such as E. coli, for example, JM109, DH5a, and HB101, or Bacillus subtilis.

Culture medium such as DMEM, MEM, RPM11640, or IMDM may be used for animal cells. The culture medium can be used with or without serum supplement such as fetal calf serum (FCS). The pH of the culture medium is preferably between about 6 and 8. Cells are typically cultured at about 30° to 40° C. for about 15 to 200 hr, and the culture medium may be replaced, aerated, or stirred if necessary.

### Transgenic Non-Human Organisms

A “transgenic non-human organism” refers to a plant or an animal, other than a human, that contains a nucleic acid construct (“transgene”) not found in a wild-type plant or animal of the same species or breed. A “transgene” as referred to herein has the normal meaning in the art of biotechnology and includes a genetic sequence which has been produced or altered by recombinant DNA or RNA technology and which has been introduced into a plant or an animal, preferably avian, cell. The transgene may include genetic sequences derived from an animal cell. Typically, the transgene has been introduced into the animal by human manipulation such as, for example, by transformation but any method can be used as one of skill in the art recognizes A transgene includes genetic sequences that are introduced into a chromosome as well as those that are extrachromosomal.

Techniques for producing transgenic animals are well known in the art. A useful general textbook on this subject is Houdebine, Transgenic animals—Generation and Use (Harwood Academic, 1997).

Heterologous DNA can be introduced, for example, into fertilized ova. For instance, totipotent or pluripotent stem cells can be transformed by microinjection, calcium phosphate mediated precipitation, liposome fusion, retroviral infection or other means, the transformed cells are then introduced into the embryo, and the embryo then develops into a transgenic animal. In one method, developing embryos are infected with a retrovirus containing the desired DNA, and transgenic animals produced from the infected embryo. In an alternative method, however, the appropriate DNAs are coinjected into the pronucleus or cytoplasm of embryos, preferably at the single cell stage, and the embryos allowed to develop into mature transgenic animals.

Another method used to produce a transgenic animal involves microinjecting a nucleic acid into pro-nuclear stage eggs by standard methods. Injected eggs are then cultured before transfer into the oviducts of pseudopregnant recipients.

Transgenic animals may also be produced by nuclear transfer technology. Using this method, fibroblasts from donor animals are stably transfected with a plasmid incorporating the coding sequences for a binding domain or binding partner of interest under the control of regulatory sequences. Stable transfectants are then fused to enucleated oocytes, cultured and transferred into female recipients.

Sperm-mediated gene transfer (SMGT) is another method that may be used to generate transgenic animals. This method was first described by Lavitrano et al. (1989).

Another method of producing transgenic animals is linker based sperm-mediated gene transfer technology (LB-SMGT). This procedure is described in U.S. Pat. No. 7,067,308. Briefly, freshly harvested semen is washed and incubated with murine monoclonal antibody mAbC (secreted by the hybridoma assigned ATCC accession number PTA-6723) and then the construct DNA. The monoclonal antibody aids in the binding of the DNA to the semen. The sperm/DNA complex is then artificially inseminated into a female.

Germline transgenic chickens may be produced by injecting replication-defective retrovirus into the subgerminal cavity of chick blastoderms in freshly laid eggs (U.S. Pat. No. 5,162,215; Bosselman et al., 1989; Thoraval et al., 1995). The retroviral nucleic acid carrying a foreign gene randomly inserts into a chromosome of the embryonic cells, generating transgenic animals, some of which bear the transgene in their germ line. Use of insulator elements inserted at the 5′ or 3′ region of the fused gene construct to overcome position effects at the site of insertion has been described (Chim et al., 1993).

Another method for generating germline transgenic animals is by using a transposon, for example the Tol2 transposon, to integrate a nucleic acid construct of the invention into the genome of an animal. The Tol2 transposon which was first isolated from the medaka fish Oryzias latipes and belongs to the hAT family of transposons is described in Koga et al. (1996) and Kawakami et al. (2000). Mini-Tol2 is a variant of Tol2 and is described in Balciunas et al. (2006). The Tol2 and Mini-Tol2 transposons facilitate integration of a transgene into the genome of an organism when co-acting with the Tol2 transposase. By delivering the Tol2 transposase on a separate non-replicating plasmid, only the Tol2 or Mini-Tol2 transposon and transgene is integrated into the genome and the plasmid containing the Tol2 transposase is lost within a limited number of cell divisions. Thus, an integrated Tol2 or Mini-Tol2 transposon will no longer have the ability to undergo a subsequent transposition event. Additionally, as Tol2 is not known to be a naturally occurring avian transposon, there is no endogenous transposase activity in an avian cell, for example a chicken cell, to cause further transposition events.

Any other suitable transposon system may be used in the methods of the present invention. For example, the transposon system may be a Sleeping Beauty, Frog Prince or Mos1 transposon system, or any transposon belonging to the tc1/mariner or hAT family of transposons may be used.

The injection of avian embryonic stem cells into recipient embryos to yield chimeric birds is described in U.S. Pat. No. 7,145,057. Breeding the resulting chimera yields transgenic birds whose genome is comprised of exogenous DNA.

Methods of obtaining transgenic chickens from long-term cultures of avian primordial germ cells (PGCs) are described in US Patent Application 20060206952. When combined with a host avian embryo by known procedures, those modified PGCs are transmitted through the germline to yield transgenic offspring.

A viral delivery system based on any appropriate virus may be used to deliver the nucleic acid constructs of the present invention to a cell. In addition, hybrid viral systems may be of use. The choice of viral delivery system will depend on various parameters, such as efficiency of delivery into the cell, tissue, or organ of interest, transduction efficiency of the system, pathogenicity, immunological and toxicity concerns, and the like. It is clear that there is no single viral system that is suitable for all applications. When selecting a viral delivery system to use in the present invention, it is important to choose a system where nucleic acid construct-containing viral particles are preferably: 1) reproducibly and stably propagated; 2) able to be purified to high titers; and 3) able to mediate targeted delivery (delivery of the nucleic acid expression construct to the cell, tissue, or organ of interest, without widespread dissemination).

In one embodiment, transfection reagents can be mixed with an isolated nucleic acid molecule, polynucleotide or nucleic acid construct as described herein and injected directly into the blood of developing avian embryos. This method is referred to herein as “direct injection”. Using such a method the isolated nucleic acid molecule, polynucleotide or nucleic acid construct of the invention is introduced into primordial germ cells (PGCs) in the embryo and inserted into the genome of the avian.

Accordingly, in the methods of the present invention an isolated nucleic acid, polynucleotide or nucleic acid construct is complexed or mixed with a suitable transfection reagent. The term “transfection reagent” as used herein refers to a composition added to the polynucleotide for enhancing the uptake of the polynucleotide into a eukaryotic cell including, but not limited to, an avian cell such as a primordial germ cell. While any transfection reagent known in the art to be suitable for transfecting eukaryotic cells may be used, the present inventors have found that transfection reagents comprising a cationic lipid are particularly useful in the methods of the present invention. Non-limiting examples of suitable commercially available transfection reagents comprising cationic lipids include Lipofectamine (Life Technologies) and Lipofectamine 2000 (Life Technologies).

The polynucleotide may be mixed (or “complexed”) with the transfection reagent according to the manufacturer's instructions or known protocols. By way of example, when transfecting plasmid DNA with Lipofectamine 2000 transfection reagent (Invitrogen, Life Technologies), DNA may be diluted in 50 μl Opit-MEM medium and mixed gently. The Lipofectamine 2000 reagent is mixed gently and an appropriate amount diluted in 50 μl Opti-MEM medium. After a 5 minute incubation, the diluted DNA and transfection reagent are combined and mixed gently at room temperature for 20 minutes.

A suitable volume of the transfection mixture may then be directly injected into an avian embryo in accordance with the method of the invention. Typically, a suitable volume for injection into an avian embryo is about 1 μl to about 3 μl, although suitable volumes may be determined by factors such as the stage of the embryo and species of avian being injected. The person skilled in the art will appreciate that the protocols for mixing the transfection reagent and DNA, as well as the volume to be injected into the avian embryo, may be optimised in light of the teachings of the present specification.

Prior to injection, eggs are incubated at a suitable temperature for embryonic development, for example around 37.5 to 38° C., with the pointy end (taglion) upward for approximately 2.5 days (Stages 12-17), or until such time as the blood vessels in the embryo are of sufficient size to allow injection. The optimal time for injection of the transfection mixture is the time of PGC migration that typically occurs around Stages 12-17, but more preferably Stages 13-14. As the person skilled in the art will appreciate, broiler line chickens typically have faster growing embryos, and so injection should preferably occur early in Stages 13-14 so as to introduce the transfection mixture into the bloodstream at the time of PGC migration.

To access a blood vessel of the avian embryo, a hole is made in the egg shell. For example, an approximately 10 mm hole may be made in the pointy end of the egg using a suitable implement such as forceps. The section of shell and associated membranes are carefully removed while avoiding injury to the embryo and it's membranes.

Following injection of the transfection mixture into the blood vessel of the avian embryo, the egg is sealed using a sufficient quantity of parafilm, or other suitable sealant film as known in the art. For example, where a 10 mm hole has been made in the shell, an approximately 20 mm square piece of parafilm may be used to cover the hole. A warm scalpel blade may then be used to affix the parafilm to the outer egg surface. Eggs are then turned over to the pointy-end down position and incubated at a temperature sufficient for the embryo to develop, such as until later analysis or hatch. The direct injection technique is further described in U.S. provisional application 61/636,331.

The non-human transgenic animals of the invention have use in breeding and food production. Once a non-human transgenic animal with an increased resistance to influenza has been produced using the method of the invention, it can be bred to select for disease resistant progeny. The disease resistant progeny, for example, disease resistant poultry, are then suitable for distribution to producers for further breeding an food production. Methods for the production of food from livestock animals are well known in the art.

### Compositions and Administration

In a preferred embodiment, a composition of the invention is a pharmaceutical composition comprising a suitable carrier. Suitable pharmaceutical carriers, excipients and/or diluents include, but are not limited to, lactose, sucrose, starch powder, talc powder, cellulose esters of alkonoic acids, magnesium stearate, magnesium oxide, crystalline cellulose, methyl cellulose, carboxymethyl cellulose, gelatin, glycerin, sodium alginate, antibacterial agents, antifungal agents, gum arabic, acacia gum, sodium and calcium salts of phosphoric and sulfuric acids, polyvinylpyrrolidone and/or polyvinyl alcohol, saline, and water.

In some embodiments, the nucleic acid construct(s) and/or nucleic acid molecules of the invention are complexed with one or more cationic lipids or cationic amphiphiles, such as the compositions disclosed in U.S. Pat. No. 4,897,355; U.S. Pat. No. 5,264,618; or U.S. Pat. No. 5,459,127. In other embodiments, they are complexed with a liposome/liposomic composition that includes a cationic lipid and optionally includes another component, such as a neutral lipid (see, for example, U.S. Pat. No. 5,279,833; U.S. Pat. No. 5,283,185; and U.S. Pat. No. 5,932,241). In other embodiments, they are complexed with the multifunctional molecular complexes of U.S. Pat. Nos. 5,837,533; 6,127,170; and 6,379,965 or, desirably, the multifunctional molecular complexes or oil/water cationic amphiphile emulsions of WO 03/093449. The latter application teaches a composition that includes a nucleic acid, an endosomolytic spermine that includes a cholesterol or fatty acid, and a targeting spermine that includes a ligand for a cell surface molecule. The ratio of positive to negative charge of the composition is between 01. to 2.0, preferably 0.5 and 1.5, inclusive; the endosomolytic spermine constitutes at least 20% of the spermine-containing molecules in the composition; and the targeting spermine constitutes at least 10% of the spermine-containing molecules in the composition. Desirably, the ratio of positive to negative charge is between 0.8 and 1.2, inclusive, such as between 0.8 and 0.9, inclusive.

Administration of a nucleic acid construct, nucleic acid molecule and/or composition may conveniently be achieved by injection into an avian egg, and generally injection into the air sac. Notwithstanding that the air sac is the preferred route of in ovo administration, other regions such as the yolk sac or chorion allantoic fluid may also be inoculated by injection. The hatchability rate might decrease slightly when the air sac is not the target for the administration although not necessarily at commercially unacceptable levels. The mechanism of injection is not critical to the practice of the present invention, although it is preferred that the needle does not cause undue damage to the egg or to the tissues and organs of the developing embryo or the extra-embryonic membranes surrounding the embryo.

Generally, a hypodermic syringe fitted with an approximately 22 gauge needle is suitable for avian in ovo administration. The method of the present invention is particularly well adapted for use with an automated injection system, such as those described in U.S. Pat. No. 4,903,635, U.S. Pat. No. 5,056,464, U.S. Pat. No. 5,136,979 and US 20060075973.

In another embodiment, the nucleic acid construct, nucleic acid molecule and/or composition of the invention is administered via pulmonary delivery, such as by inhalation of an aerosol or spray dried formulation. For example, the aerosol may be administered by an inhalation device or nebulizer (see for example U.S. Pat. No. 4,501,729), providing rapid local uptake of the nucleic acid molecules into relevant pulmonary tissues. Solid particulate compositions containing respirable dry particles of micronized nucleic acid compositions can be prepared by grinding dried or lyophilized nucleic acid compositions, and then passing the micronized composition through, for example, a 400 mesh screen to break up or separate out large agglomerates. A solid particulate composition comprising the nucleic acid compositions of the invention can optionally contain a dispersant which serves to facilitate the formation of an aerosol as well as other therapeutic compounds. A suitable dispersant is lactose, which can be blended with the nucleic acid compound in any suitable ratio, such as a 1 to 1 ratio by weight.

Nebulizers are commercially available devices which transform solutions or suspensions of an active ingredient into a therapeutic aerosol mist either by means of acceleration of a compressed gas, typically air or oxygen, through a narrow venturi orifice or by means of ultrasonic agitation. Suitable formulations for use in nebulizers comprise the active ingredient in a liquid carrier in an amount of up to 40% w/w preferably less than 20% w/w of the formulation. The carrier is typically water or a dilute aqueous alcoholic solution, preferably made isotonic with body fluids by the addition of, for example, sodium chloride or other suitable salts. Optional additives include preservatives if the formulation is not prepared sterile, for example, methyl hydroxybenzoate, anti-oxidants, flavorings, volatile oils, buffering agents and emulsifiers and other formulation surfactants. The aerosols of solid particles comprising the active composition and surfactant can likewise be produced with any solid particulate aerosol generator. Aerosol generators for administering solid particulate therapeutics to a subject produce particles which are respirable, as explained above, and generate a volume of aerosol containing a predetermined metered dose of a therapeutic composition at a rate suitable for human administration.

A nucleic acid construct, nucleic acid molecule and/or composition of the invention can also be added to animal feed or drinking water. It can be convenient to formulate the feed and drinking water compositions so that the animal takes in a therapeutically appropriate quantity along with its diet. It can also be convenient to present the composition as a premix for addition to the feed or drinking water.

### Therapeutic and Prophylactic Methods

The present invention utilises the isolated nucleic acid molecules and nucleic acid constructs of the invention for the treatment and prevention of influenza in a subject. In one embodiment, the therapeutic and/or prophylactic methods of the invention comprise administering the isolated nucleic acid molecule of the invention, the nucleic acid construct of the invention, the vector of the invention, the cell of the invention, or the composition of the invention to the subject.

In some instances it may be desirable to combine the administration of the inventive nucleic acid molecules, constructs, vectors or compositions (“the compounds of the invention”) described herein with one or more other anti-viral agents in order to inhibit, reduce, or prevent one or more symptoms or characteristics of infection. In certain embodiments of the invention, the compounds of the invention are combined with one or more other antiviral agents such as NA inhibitors, M inhibitors, etc. Examples include amantadine or rimantadine and/or zanamivir, oseltamivir, peramivir (BCX-1812, RWJ-270201) Ro64-0796 (GS 4104) or RWJ-270201. However, the administration of the compounds of the invention may also be combined with one or more of any of a variety of agents including, for example, influenza vaccines (e.g., conventional vaccines employing influenza viruses or viral antigens as well as DNA vaccines) of which a variety are known.

The compounds of the invention may be present in the same mixture as the other agent(s) or the treatment regimen for an individual includes both the compounds of the invention and the other agent(s), not necessarily delivered in the same mixture or at the same time. Thus, as used herein, the term “combination” is not intended to indicate that compounds must be present in, or administered to a subject as, a single composition of matter, e.g., as part of the same dosage unit (e.g., in the same aerosol formulation, particle composition, tablet, capsule, pill, solution, etc.) although they may be. Instead, in certain embodiments of the invention the agents are administered individually but concurrently. As used herein the term “coadministration” or “concurrent administration” of two or more compounds is not intended to indicate that the compounds must be administered at precisely the same time. In general, compounds are coadministered or administered concurrently if they are present within the body at the same time in less than de minimis quantities.

The inventive therapeutic protocols described herein involve administering an effective amount of compound of the invention, simultaneously with, or after exposure to influenza virus. For example, uninfected individuals may be treated with an inventive composition prior to exposure to influenza; at risk individuals (e.g., the elderly, immunocompromised individuals, persons who have recently been in contact with someone who is suspected, likely, or known to be infected with influenza virus, etc.) can be treated substantially contemporaneously with exposure, e.g., within 2 hours or less following exposure. In other embodiments a subject is treated at a later time, e.g., within 2-12, 12-24, 24-36, or 36-48 hours, following a suspected or known exposure. The subject may be symptomatic or asymptomatic. In certain embodiments the subject is protected by administration of a compound of the invention up to 48 hours, up to 24 hours, up to 12 hours, up to 3 hours, etc., before an exposure. Of course individuals suspected or known to be infected may receive inventive treatment at any time.

Influenza viruses infect a wide variety of species in addition to humans. The present invention includes the use of the inventive compositions for the treatment of nonhuman species, particularly species such as chickens, ducks, turkeys, swine, and horses. For the treatment and prevention of influenza in livestock animals, it may be desirable to formulate the inventive nucleic acid molecules, constructs, vectors or compositions into an animal feed or in drinking water.

### EXAMPLES

### Example 1

### Identification of NS1-NEP Target Region

The present inventors have identified a region of the influenza genome which overlaps the NS1 and NEP open reading frames in genome segment 8. Within this region, the inventors have advantageously identified a 23 nucleotide sequence that is conserved across many influenza virus isolates, thus making it an excellent target for molecules that induce RNA interference in a cell. The sequence of the 23 nucleotide conserved region is provided as SEQ ID NO:1.

Provided in SEQ ID NOs:2 to 6 are 19-mer oligonucleotides complementary to target sequences within the conserved 23 nucleotide region of NS1-NEP. Nucleic acid molecules comprising a double-stranded region complementary to a target sequence comprising any one of SEQ ID NOs:2 to 6 share the common structural and functional capability of being able to inhibit the expression of a target sequence comprising SEQ ID NO:12. Expression constructs comprising an shRNA targeting the NS1-NEP conserved region under control of the U64 promoter (SEQ ID NO:7) and H1 promoter (SEQ ID NO:8) were also prepared, along with a multi-warhead construct expressing shRNA targeting NS1-NEP, PB1-2257 and NP-1498 (SEQ ID NO:11).

### Example 2

### In Vitro Silencing of Influenza PR8 in MDCK Cells by shRNA

The inventors tested the ability of an shRNA comprising a sequence complementary to SEQ ID NO:2 to induce RNA interference and inhibition of viral replication in MDCK cells infected with the PR8 strain of influenza virus. In this experiment, the shRNA sequence was expressed from the U64 promoter (SEQ ID NO:7).

MDCK cells were freshly passed on the day prior to experiments so that the cells were healthy and not over confluent. The cells were trypsinised to remove them from the flask, counted and aliquoted into 1.5 ml microcentrifuge tubes at 1×10⁶ cells per tube. Cells were pelleted at 4000 rpm for 3 minutes in a bench-top microcentrifuge.

MDCK cells were cultured in Earls Modified Eagle's Medium containing 10% Foetal Calf Serum (FCS), 10 mM Hepes, 2 mM glutamine, supplemented with penicillin (100 U/ml) and streptomycin (100 μg/ml) at 37° C. in a humidified atmosphere containing 5% CO₂. Logarithmic phase cells were trypsinized, counted and aliquoted into 1.5 million cells for each electroporation. Cells were resuspended in 100 μl of electroporation solution T (Amaxa), mixed with 2.5 μg of plasmid DNA and electroporated using program T20 of the Amaxa Nucleofector. Cells were plated out in a 24 well plate at 250 000 cells per well in duplicate for each infection.

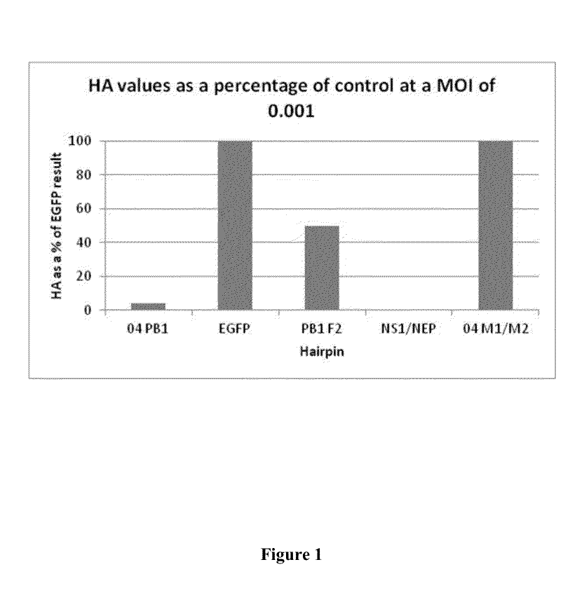

After incubation for 31 hours, medium was removed from the cells and 200 μl of influenza PR8 virus diluted in Viral Growth Media was added. The Viral Growth Media comprised EMEM, 0.3% bovine serum albumen, HEPES, glutamine, penicillin/streptomycin and 5 μg/ml trypsin. Infections were performed in duplicate. The cells were incubated at 37° C. for 1 hour. Virus was removed from wells and 500 μl of Viral Growth Medium added to each well. After 66 hours incubation, the supernatants were removed and hemagglutination assays were performed. Cells were removed in RLT buffer and used in real time PCR assays. The results of the hemagglutination assays are provided in Table 1 and FIG. 1.

TABLE 1
Results of Hemaglutination Assays

| | EGPN1 | | |
|---|---|---|---|
| MOI (Multiplicity | Transfection control | | |
| Of Infection) | (−ve) | 04PB1 (+ve) | 04NS1/NEP |
| 0.01 | 64 | 16 | 8 |
| 0.01 | 32 | 16 | 16 |
| 0.001 | 64 | 0 | 0 |
| 0.001 | 32 | 4 | 0 |

### Example 3

### Quantitative PCR

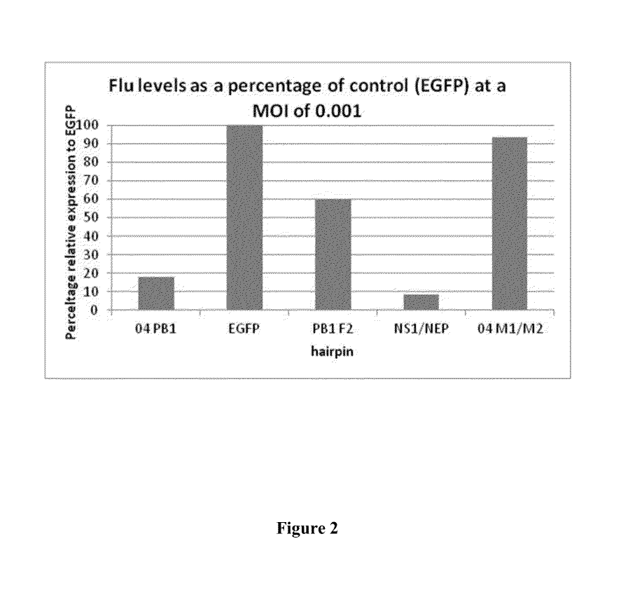

Total RNA was isolated from cells described above in Example 2 using a Qiagen RNeasy kit and viral mRNA was quantified by real time PCR with specific primers for Influenza A M gene (Heine et al., 2007) using an ABI 7700 sequence detection system (Applied Biosystems). A primer/probe mix was prepared by mixing together equal volumes of primers IVA-D161M (18 μM) and IVA-D162M (18 μM) and probe IVA-Ma (FAM) (5 μM) (Heine et al., 2007). Real-time reverse transcription-PCR (RRT-PCR) reactions were set up in 25 μl volumes comprising 5.75 μl nuclease-free water; 12.5 μl TaqMan 2× Universal PCR master mix no AmpErase UNG; 0.625 μl 40× Multiscribe and RNase inhibitor mix; 4.125 μA test primer probe mix; and 2 μA of total RNA. Reactions were run in a real-time PCR thermocycler with the following parameters: 30 min at 48° C. (reverse transcription), 10 min at 95° C. (hot-start Taq polymerase activation), and 45 cycles of 15 sec at 95° C. and 1 min at 60° C. (target amplification). PCR results were analysed were made relative to the EGFP control sample and represented as a percentage of this control. Results in FIG. 2 show that NS1/NEP shRNA reduced viral RNA levels by greater than 90% compared to the EGFP control plasmid.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Balciunas et al. (2006) PLoS Genet. 10:e169.
Bosselman et al. (1989) Science, 243:533-534.
Chim et al. (1993) Cell, 74:504-514.
Dua et al. (2011) Mo Ther, 19:1676-1687.
Heine et al. (2007) Avian Dis. 51:370-372.
Kawakami et al. (2000) Proc Natl Acad Sci USA, 97:11403-11408.
Lavitrano et al. (1989) Cell, 57:717-723.
Liu et al. (2007) Nucl Acids Res. 35:5683-5693.
Thoraval et al. (1995) Transgenic Research 4:369-36.

1. An isolated nucleic acid molecule comprising a double-stranded region, the double-stranded region comprising a sequence of nucleotides complementary to a target sequence, wherein the target sequence is at least 90% identical to any one of SEQ ID NOs:2 to 6.

2. The isolated nucleic acid molecule of claim 1, wherein the target sequence is identical to any one of SEQ ID NOs:2 to 6.

3. The isolated nucleic acid molecule of claim 1, wherein the double-stranded region is 19 to 23 nucleotides in length.

4. The isolated nucleic acid molecule of claim 1 which comprises RNA.

5. The isolated nucleic acid molecule of claim 4 which is an siRNA, shRNA, eshRNA, or miRNA.

6. A nucleic acid construct encoding the isolated nucleic acid molecule of claim 1.

7. An expression vector comprising the nucleic acid construct of claim 6.

8. (canceled)

9. A cell comprising the nucleic acid construct of claim 6.

10.-11. (canceled)

12. A composition comprising the isolated nucleic acid molecule of claim 1.

13. A method of treating or preventing influenza in a subject, the method comprising administering to the subject the isolated nucleic acid molecule of claim 1.

14. The method of claim 13, wherein the subject is a non-human animal.

15. The method of claim 14, wherein the non-human animal is an avian.

16. (canceled)

17. A non-human transgenic organism comprising the nucleic acid construct of claim 6.

18. (canceled)

19. The non-human transgenic organism of claim 17 which is a non-human transgenic animal.

20. The non-human transgenic animal of claim 19 which is an avian.

21.-23. (canceled)

24. A method of reducing the level of expression of influenza A virus NS1 and NEP genes in a cell, the method comprising introducing into the cell the isolated nucleic acid molecule of claim 1.

25-26. (canceled)

27. A method of making a transgenic non-human animal, the method comprising:
(i) introducing a first nucleic acid comprising a transposon into a cell, wherein the nucleic acid encodes the isolated nucleic acid of claim 1,
(ii) introducing a second nucleic acid encoding a transposase into the cell,
(ii) selecting a transgenic cell comprising the first nucleic acid in the genome of the cell,
(iii) regenerating a transgenic non-human animal from the cell, and
(iv) breeding the transgenic non-human animal.

28-30. (canceled)

31. A composition comprising the nucleic acid construct of claim 6.

32. A method of treating or preventing influenza in a subject, the method comprising administering to the subject the nucleic acid construct of claim 6.

33. A method of reducing the level of expression of influenza A virus NS1 and NEP genes in a cell, the method comprising introducing into the cell the nucleic acid construct of claim 6.


SEQUENCE LISTING (12 sequences)

SEQ ID NO: 1 - DNA, 23 bp, Artificial Sequence
  Synthetic nucleic acid sequence
ttgaatggaatgataacacagtt

SEQ ID NO: 2 - DNA, 19 bp, Artificial Sequence
  Synthetic nucleic acid sequence
aatggaatgataacacagt

SEQ ID NO: 3 - DNA, 19 bp, Artificial Sequence
  Synthetic nucleic acid sequence
atggaatgataacacagtt

SEQ ID NO: 4 - DNA, 19 bp, Artificial Sequence
  Synthetic nucleic acid sequence
gaatggaatgataacacag

SEQ ID NO: 5 - DNA, 19 bp, Artificial Sequence
  Synthetic nucleic acid sequence
tgaatggaatgataacaca

SEQ ID NO: 6 - DNA, 19 bp, Artificial Sequence
  Synthetic nucleic acid sequence
ttgaatggaatgataacac

SEQ ID NO: 7 - DNA, 355 bp, Artificial Sequence
  Synthetic nucleic acid sequence
gtcgacgaattgtgggacggcggaagacgggctcccgccccgcccctatatgcaaagcagagaacttcccgccgtgcaccgcgcgcaatcggaagagaatttgggcacttcagacccaaaaaaaaacccaaaacttctgcgaaaaagaaagaatctcagcggagtaaatagggatttttgttaaagaggtgataccagaagaagaaatatgcaaatacaacgccagctcaccgctacttaaaaatcatgatataatagaggcttaaatactgtccgagagacactggggtttaatggaatgataacacagtttcaagagaactgtgttatcattccattttttttggaactcgag

SEQ ID NO: 8 - DNA, 334 bp, Artificial Sequence
  Synthetic nucleic acid sequence
gtcgacggcaccagacccattcacacccccaaccccccaagctgaggctgcccgtggccccatccatggccttggggacctcgacggatggggcacgcgcaactcctgcaccctcccaaccgctccgtataccgccgctccctcactcccagcacgaggcggggcggggccgccgtcattagcatacaccgcagagcatgccgggtaactcgccctactctaaaagggttccggaaccagagctgtatataggcacttccacaaagtgcgtaatggaatgataacacagtttcaagagaactgtgttatcattccattttttttggaactcgag

SEQ ID NO: 9 - DNA, 19 bp, Artificial Sequence
  Synthetic nucleic acid sequence
gatctgttccaccattgaa

SEQ ID NO: 10 - DNA, 21 bp, Artificial Sequence
  Synthetic nucleic acid sequence
atcttatttcttcggagacaa

SEQ ID NO: 11 - DNA, 1147 bp, Artificial Sequence
  Synthetic nucleic acid sequence
gtcgacgaattgtgggacggcggaagacgggctcccgccccgcccctatatgcaaagcagagaacttcccgccgtgcaccgcgcgcaatcggaagagaatttgggcacttcagacccaaaaaaaacccaaaacttctgcgaaaaagaaagaatctcagcggagtaaatagggatttttgttaaagaggtgataccagaagaagaaatatgcaaatacaacgccagctctgagctacttaaaaatcatgatataatagaggcttaaatactgtccgagagacactggggtttgatctgttccaccattgaattcaagagattcaatggtggaacagatcttttttggaactcgaccagacagacgtcaggctttctaagcctggactgagtaagaccggaagagctccacagcactctgagtgcgcagagaccgcgcgtacagcgcacagccgcgcggccgctccttcaggcactgccgacgacagcccaggcggaggtcctgagcgccggcgctaaatttgcataaagaactacccaggagccctcgcgcgcggaaacgggcaaaaaggggcttctaatatggaaatattacgccgaatcgcgtcacaaatcggctaagcgggcctaagagttaacaagatgtgctattaagcggagccttttggtggaaagaaacggaacagttgttgtgtcctaaaagaacttgcagaatgaacctttaaataccgcagtctcgatgctcttagtcatcttatttcttcggagacaattcaagagattgtctccgaagaaataagatttttttggaactcgacggcaccagacccattcacacccccaaccccccaagctgaggctgcccgtggccccatccatggccttggggacctcgacgggtggggcacgcgcaactcctgcaccctcccaaccgctccgtataccgccgctcccccactcccagcacgaggcggggcggggccgccgtcattagcatacaccgcagagcatgccgggtaactcgccctactctaaaagggttccggaaccagagctgtatataggcacttccacaaagtgcgtaatggaatgataacacagtttcaagagaactgtgttatcattccattttttttggaactcgacctcg

SEQ ID NO: 12 - DNA, 15 bp, Artificial Sequence
  Synthetic nucleic acid sequence
atggaatgataacac